(19) United States
(12) Patent Application Publication
(10) Pub. No.: US 2019/0167133 A1
(43) Pub. Date: Jun. 6, 2019

(54) WIRELESS BIOLOGICAL INTERFACE PLATFORM

(71) Applicant: Pluri, Inc., Tustin, CA (US)

(72) Inventor: David Frausto Pena, Chino Hills, CA (US)

(21) Appl. No.: 16/314,344

(22) PCT Filed: Jun. 15, 2017
(86) PCT No.: PCT/US2017/037641

§ 371 Date: Dec. 28, 2018

Related U.S. Application Data
(60) Provisional application No. 62/350,396, filed on Jun. 15, 2016.

(51) Int. Cl. A61B 5/04; A61B 5/00

(52) U.S. Cl. CPC A61B 5/04; A61B 5/0031; A61B 5/686; A61B 2560/0219; A61B 2503/40; A61B 2560/0209; A61B 2560/045

(57) ABSTRACT

A biological interface platform may include a WGPU customized for biological implantation in a living organism. The WGPU may be connected to modular probes, which may also be implanted and integrated together via software. The biological interface platform may be designed for the recording and/or transmission of biological information to and/or from a subject via a wireless interface and integration of that information across a software workspace.

### CROSS-REFERENCE TO RELATED APPLICATION

This application claims the benefit of U.S. Provisional Patent Application Ser. No. 62/350,396 filed on Jun. 15, 2016. The subject matter of this earlier filed application is hereby incorporated by reference in its entirety.

### FIELD

The present invention generally relates to biological interfaces, and more particularly, to a wireless biological interface platform that includes a wireless general processing unit (WGPU), modular probes connected to the WGPU, and software that integrates the WGPU and modular probes together.

### BACKGROUND

Gathering and transmitting biological information in real-time from and to a subject is a general problem for those who experiment with animals and humans. The current state of the art for solving this problem is to tether the subject with wires to a device specialized to receive or transmit a particular type of biological signal. For instance, a neuroscientist attempting to record gross brain activity (i.e., the signal) using electroencephalography would connect a human or animal subject with electrodes to a device specialized for recording only these signals. If the same researcher wanted to simultaneously transmit information to the subject's brain electrically or optically, he or she would need a distinct device also tethered to the subject. This sort of setup poses several problems operationally.

Current trends in behavioral science have progressed to measuring and modifying natural behaviors, and the most common method of physical tethering between the subject and the recording or transmitting devices is behaviorally limiting. Tethering a subject with wires limits the mobility of the subject and exposes the apparatus to damage from the subject when the behavior of the subjects is not predictable, as is the case with animal subjects. Also, physical proximity to the recording or transmitting devices limits the number of subjects an experimenter can examine at once. This limitation increases the length of time required to complete an experiment. A simple animal experiment requires ten animals per group to resolve an effect for a single publication figure. A minimum of two groups are required to make a comparison—i.e., treatment and control. A small publication generally contains three figures. Therefore, a researcher would need at least sixty animals for a small publication. This number assumes all animals are retained for the publication, which rarely happens.

The number of possible subjects to run in a given time is limited by the equipment setup. Specifically, tethered options generally are limited in the number of subjects they can run at a time, and an experiment requiring the incorporation of two devices is limited by the device with the least possible number of connections per subject. Current standard optical stimulation devices can connect a single subject per device. Even over a short experiment where measurements are taken for a week per subject, running a single subject per day would take an estimated 1.14 years to complete the experiment. This number quickly increases if the experimenter wishes to increase the number of subjects and/or monitoring time length for each subject.

To decrease the length of time to complete an experiment, researchers will generally purchase more devices to run simultaneously. However, each device is prohibitively expensive, so a researcher is faced with the problem of taking longer to publish or investing several thousand dollars in specialized devices that may not be scientifically useful in several years. Accordingly, a reasonably priced device that allows for free movement, multiple subjects, and flexibility in biological recording and signaling may be beneficial.

### SUMMARY

Certain embodiments of the present invention may provide solutions to the problems and needs in the art that have not yet been fully identified, appreciated, or solved by current biological interface technologies. For example, some embodiments pertain to a novel platform that solves many or all operational issues currently imposed on behavioral scientists based on advancements in mobile technology and biological probes. The platform in some embodiments includes a wireless general processing unit (WGPU) customized for biological implantation in a living organism. The WGPU may be connected to modular probes, which may also be implanted and integrated together via software. The platform in some embodiments is designed for the recording and/or transmission of biological information to and/or from a subject via a wireless interface and integration of that information across a software workspace.

In an embodiment, a system includes a plurality of modular probes and at least one WGPU operably connected to the plurality of modular probes. The system also includes at least one adapter for respective external hardware. The at least one behavioral adapter is configured to relay information from the respective external hardware. The system also includes a computing system running a biological interface application configured to process input from the at least one WGPU and the at least one adapter. The biological interface application is configured to coordinate digital information from the external hardware received via the at least one adapter with activity of the at least one WGPU configured with one or more of the plurality of modular probes. The biological interface application is also configured to receive and process the information relayed from the at least one adapter.

In another embodiment, a WGPU includes a plurality of physical ports on an underside of the WGPU and a biocompatible enclosure that elevates the WGPU to allow access to the physical ports. The biocompatible enclosure protects electronic components of the WGPU from exposure to interstitial fluid while reducing immune response from a subject when implanted.

In yet another embodiment, an adapter includes a step-down regulator configured to step-down a voltage of a signal from external hardware to a lower voltage and a microcontroller configured to receive and process the stepped-down signal. The adapter also includes a USB-to-serial adapter configured to receive communications from the microcontroller and relay the communications to a biological interface application of a computing system.

### BRIEF DESCRIPTION OF THE DRAWINGS

In order that the advantages of certain embodiments of the invention will be readily understood, a more particular description of the invention briefly described above will be rendered by reference to specific embodiments that are illustrated in the appended drawings. While it should be understood that these drawings depict only typical embodiments of the invention and are not therefore to be considered to be limiting of its scope, the invention will be described and explained with additional specificity and detail through the use of the accompanying drawings, in which:

FIG. 1 is an architectural diagram illustrating a wireless biological interface platform, according to an embodiment of the present invention.

FIG. 2 illustrates a biological interface platform, according to an embodiment of the present invention.

FIG. 3 illustrates top, perspective, and side perspective views of a biocompatible enclosure for a WGPU, according to an embodiment of the present invention.

FIG. 4 is a perspective view illustrating a WGPU separated from a biocompatible enclosure, according to an embodiment of the present invention.

FIG. 5 is a flowchart illustrating a process for low power standby operation of a WGPU, according to an embodiment of the present invention.

FIG. 6 is a flowchart illustrating a process for setting of parameters of firmware of a WGPU via biological interface software, according to an embodiment of the present invention.

FIG. 7A is an exploded perspective view illustrating an optical stimulation probe, according to an embodiment of the present invention.

FIG. 7B illustrates side, top, and perspective views of the biocompatible enclosure of FIG. 7A, according to an embodiment of the present invention.

FIG. 8 illustrates a system for recording signals from a subject, according to an embodiment of the present invention.

FIG. 9 illustrates a biological interface system that includes a more detailed view of a behavioral apparatus adapter, according to an embodiment of the present invention.

FIG. 10 is a block diagram of a computing system configured to run biological interface software, according to an embodiment of the present invention.

### DETAILED DESCRIPTION OF THE EMBODIMENTS

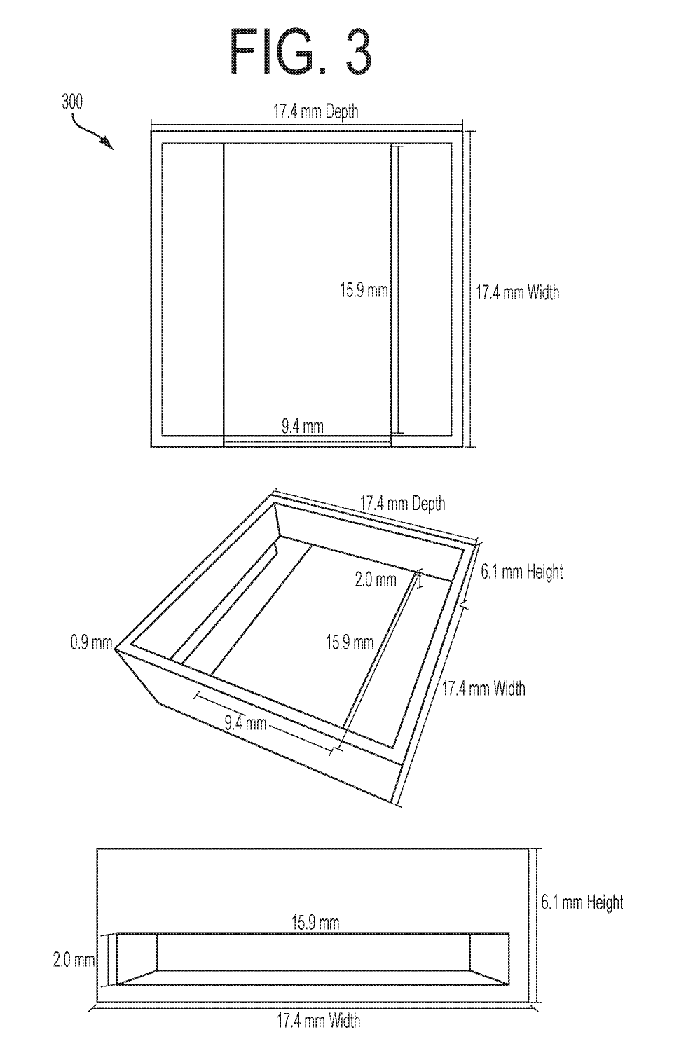

Some embodiments of the present invention pertain to a novel platform that solves many or all operational issues currently imposed on behavioral scientists based on advancements in mobile technology and biological probes. The platform in some embodiments includes a WGPU customized for biological implantation in a living organism. The WGPU may be connected to modular probes, which may also be implanted and integrated together via software. See, for example, FIGS. 3-8. The platform in some embodiments is designed for the recording and/or transmission of biological information to and/or from a subject via a wireless interface and integration of that information across a software workspace.

The platform may also include an externally accessible power module, a computer for the user, platform software, and a behavioral apparatus adapter. The system in some embodiments is a platform in the sense that probes, WGPUs, and behavioral adapters are modular and can be configured to dynamically interact with one another based on the user's desired interaction between the devices. Additionally, users may be able to add custom-built or outside manufacturer built devices to increase the functionality of the system. In this sense, the system may be a starting platform from which users can build wireless biological interfaces. These interfaces may be used in any suitable experiment using any model organism including, but not limited to, primates, fish, rodents, and mollusks. Furthermore, a wireless interface with platform software can be used outside the confines of experimentation and for long-term implantation in a human subject for therapeutic or medical purposes including, but not limited to, the long-term control of limbs, cessation of seizures, remediation of pain, implantation of artificial memories, remediation of post-traumatic stress disorder symptoms, and/or artificially enhanced learning.

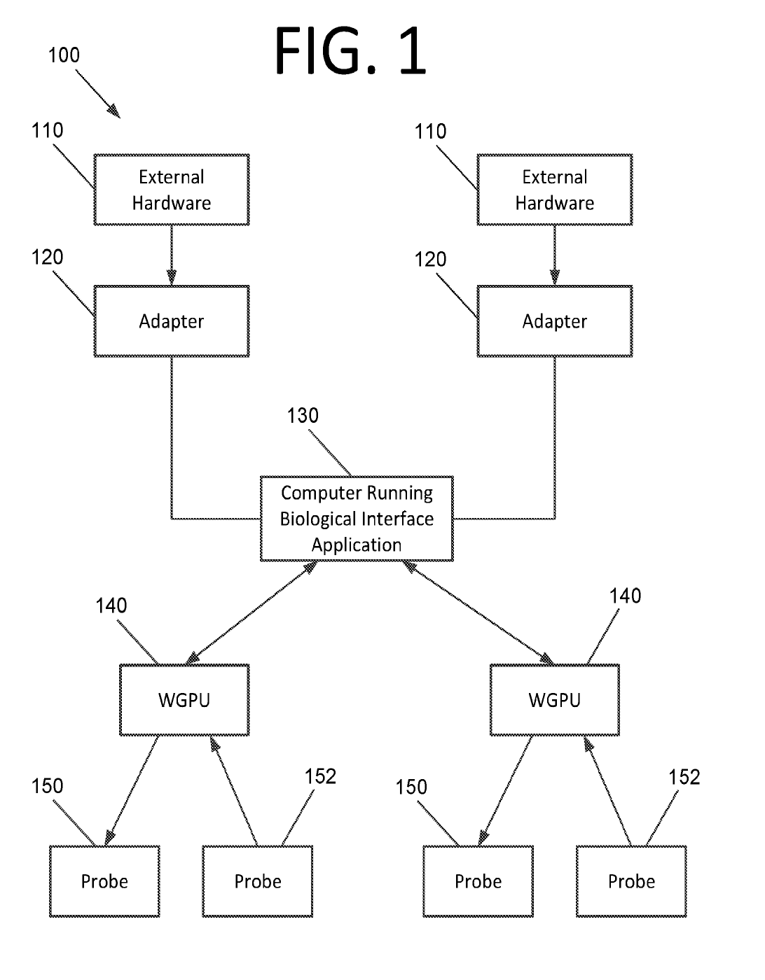

FIG. 1 is an architectural diagram illustrating a wireless biological interface platform 100, according to an embodiment of the present invention. Biological interface platform 100 includes external hardware 110 (e.g., a video monitor, an operant chamber such as the PhenoTyper™ by Noldus™, or any other suitable external hardware without deviating from the scope of the invention), adapters 120 for each piece of external hardware 110, a computer 130 running a biological interface application, WGPUs 140, recording probes 150, and transmitting probes 152. The biological interface application coordinates digital information from external hardware 110 received via adapters 120 hardware with the activity of WGPUs 140 configured with associated recording probes 150 and/or transmitting probes 152.

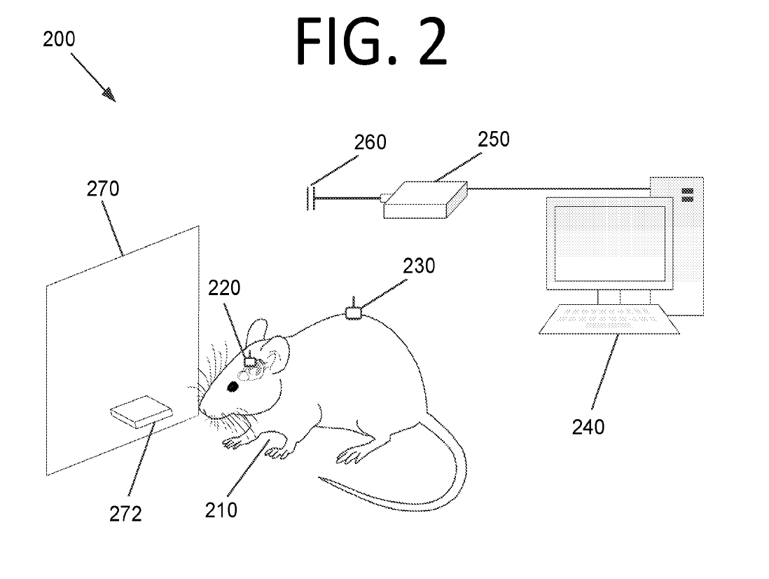

WGPUs 140 may be configured at run-time in some embodiments using workspace software (e.g., the application running on computer 130) to relay and/or transmit information to connected probes 150, 152 and back to the user for viewing, analysis, and/or action. See, for example, FIG. 2. The application may communicate through standard wireless protocols and control multiple WGPUs 140 at once, allowing multiple subjects to run simultaneously without an increase in cost.

Biological interface platform 100 includes adapters 120 for integration with current standard operant behavioral hardware (i.e., external hardware 110). Commonly used behavioral hardware typically uses varying digital out voltages depending on the manufacturer, normally ranging from 5 to 32 volts. To integrate with existing hardware, adapters 120 can receive variable voltage digital signals and relay digital information from external hardware 110 to the biological interface application.

FIG. 2 illustrates a biological interface platform 200, according to an embodiment of the present invention. In FIG. 2, a mouse 210 has biological probes 220 (e.g., optical probes) and an external WGPU 230. An experimenter may control operation of WGPU 230 using biological interface software running on a computer 240. In this embodiment, biological probes 220, WGPU 230, and computer 240 communicate wirelessly. WGPU 230 untethers subjects, allowing for free movement and real-time data acquisition.

WGPU 230 manages data being received by biological probes 220 and activation commands from the user. This connection can be dynamically modulated by the subject or the environment via connection to a behavioral apparatus adapter 250 that receives digital input/output (I/O) 260 from behavioral apparatus 270. For instance, when mouse 210 presses lever 272, this information may be relayed from behavioral apparatus 270 to behavioral apparatus adapter 250, which then relays the information to computer 240 for processing.

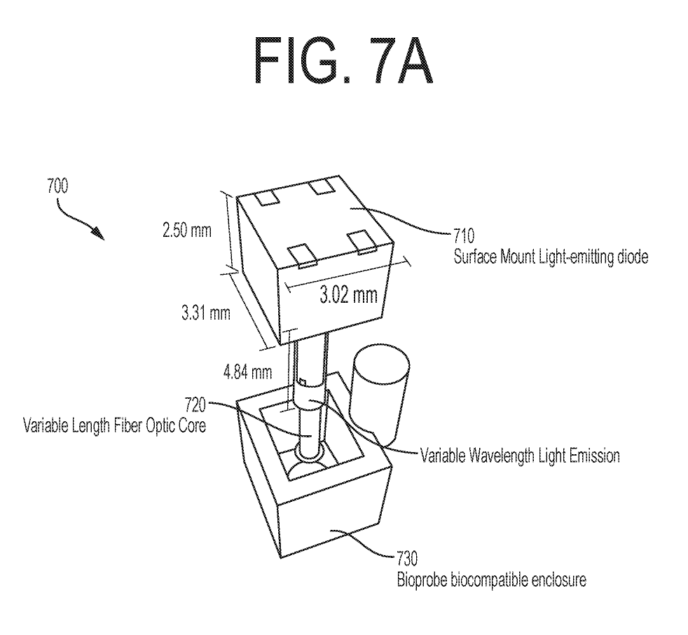

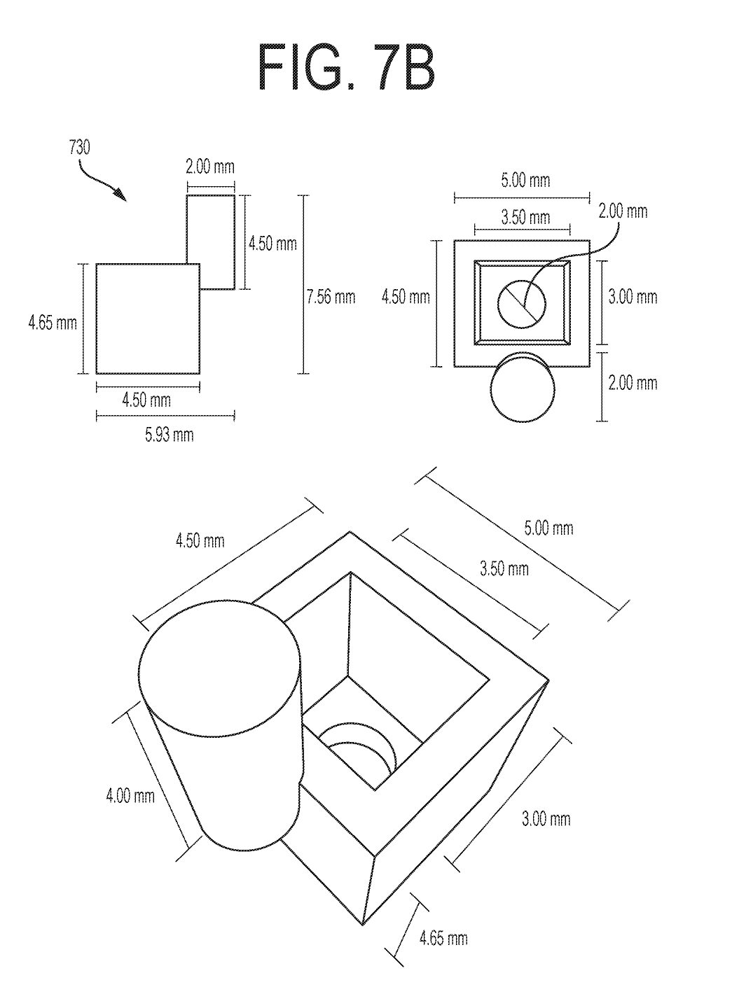

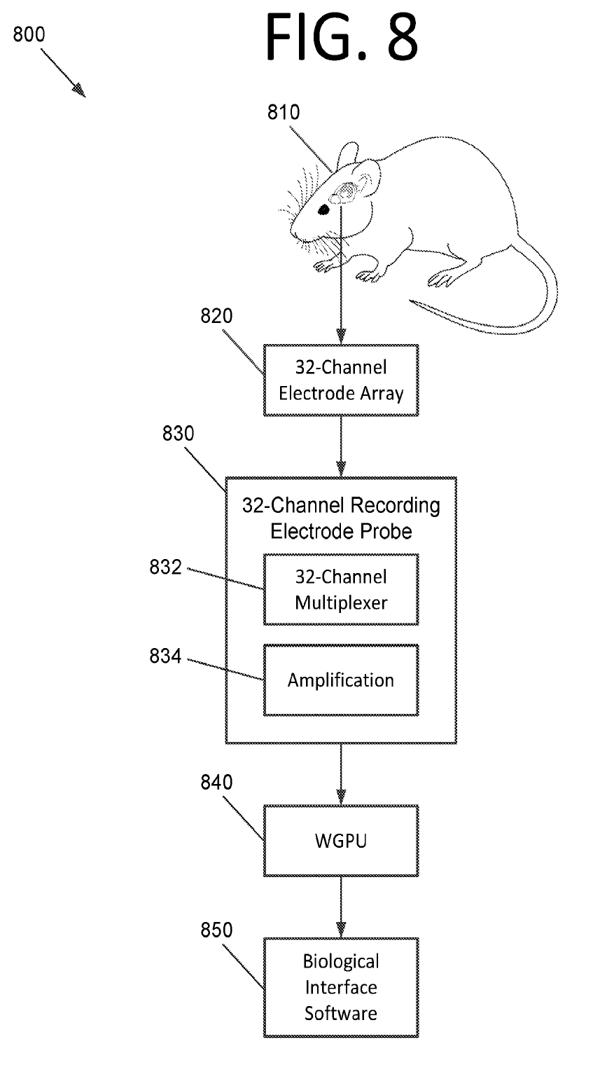

An example usage of the system in some embodiments may be as follows. A neuroscientist interested in memory formation might be interested in the neuronal ensembles responsible for a certain memory and in addition would like to artificially manipulate those memories. To artificially manipulate the firing of ensembles of neurons, an experimenter would first need to make their target cells photosensitive by a number of commercially available options, including infusion of a commercially available vector packaged with a photosensitive channel or the breeding of transgenic animal lines for expression of photosensitive channel expression. For neuronal excitation, the most common option is the expression of channelrhodopsin-2 (ChR2) on the surface of a target population of neurons. ChR2s are light-gated ion channels with maximal excitation at 470 nm (blue light). Once expression in the target regions is achieved (e.g., in a portion of cortex sensitive to a particular frequency of sound (AUD) and a portion of the limbic system whose activity is tightly correlated with fear responses (CA)), the experimenter can then implant two optic bioprobes. See FIGS. 7 and 8. The light emission of the optic bioprobes may be produced by a surface mount light emitting diode (LED) concentrated into a relatively small 250 micrometer diameter fiber optic core, the emission of which matches the optimal excitation of the target opsin ChR2.

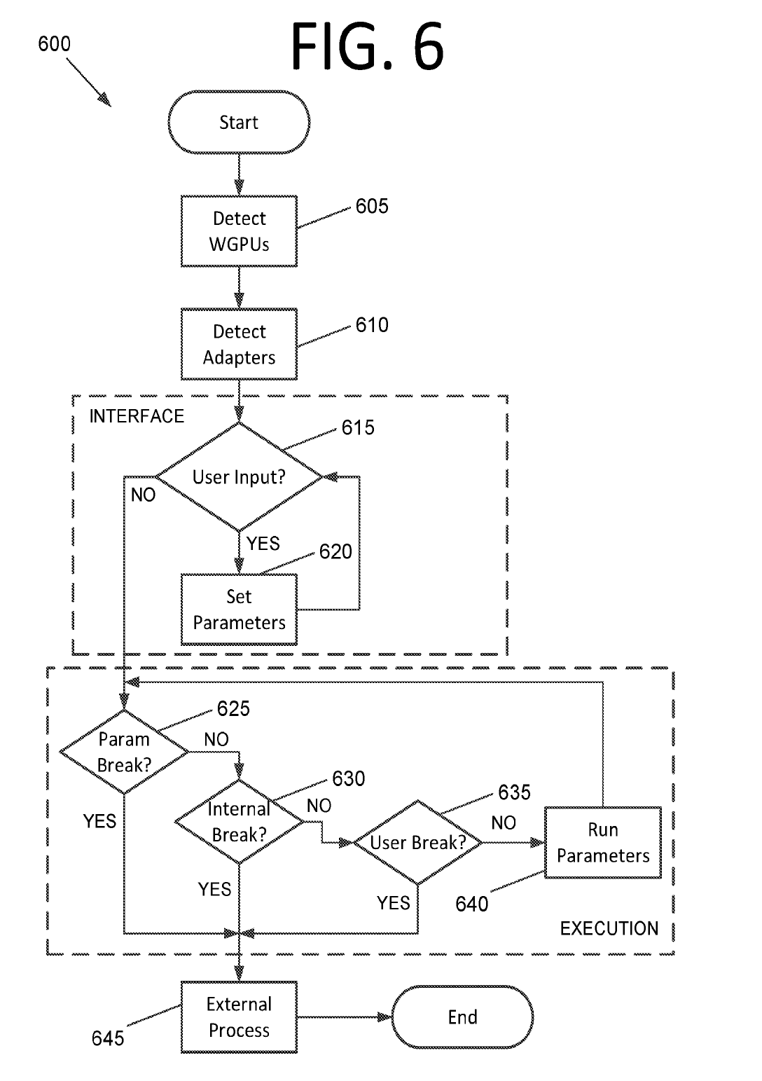

The fiber optic core is targeted at the two regions of interest, and the entire probe is fastened securely to the skull using polyacrylamide. The emission of the optic bioprobes is controlled and powered through direct subcutaneous interface with the WGPU, which itself is powered by a modular 3V battery power source. The pattern of activation of the bioprobes is set programmatically by the experimenter using biological interface platform software set to control the ports of the WGPU depending on the experimental needs during the experiment. See FIG. 6.

In this example, the experimenter may wish to elicit sequential activation of the bioprobes located in the AUD (associated with frequency X) then CA (associated with fear response) in some context (Context A). Following the hypothesis that sequential firing of neuronal ensembles informationally associates their behavior, the mentioned procedure should artificially associate frequency X with CA fear behavior. The experimenter can then test the validity of this hypothesis by examining whether an association has been formed. To test the validity, the experimenter can place the same experimental subject in a new context (Context B), play natural sound frequency X to the subject, and assess whether fear behavior is present. The experimenter can then verify that the behavior is frequency X-specific and then randomize placement in AUD in additional subjects to show the association is artificial and not an inherent property of any particular frequency. This would demonstrate that the experimenter has generated artificial memory of frequency X association with fear response.

The bioprobes, WGPU, and biological interface platform software in some embodiments offer a kind of dynamic flexibility that renders them a platform for a very large number of use cases. This example usage solves several hurdles the researcher would have stumbled upon by using conventional means. Current common practice of each probe would require its own physical tethering to an external signaling or recording device, prohibiting the movement of the experimental subject. If the probes were implanted on the skull of a small animal, like a mouse or rat, probes for the tethered approaches would not likely be able to physically fit at the same time on the surface of the skull. If the probes are able to fit, the experimenter then must have purchased two separate control devices for the two separate probes and custom-integrated each probe with his or her experimental apparatus separately. Given the cost of purchasing two set-ups, the experimenter would be limited to running very few subjects simultaneously. This fact extends the length of time necessary to run preliminary subjects to determine whether the experiment will be successful. If it the experiment is feasible, the experimenter would then need to increase the number of subjects to reach a desired level of statistical power for publication. On the other hand, the platform of some embodiments uses a single setup for all components necessary for the experiment, the subject would be un-tethered, and multiple subjects could be run simultaneously.

A WGPU in some embodiments may include a general processing unit with wireless capability placed into a biocompatible enclosure. FIG. 3 illustrates top, perspective, and side perspective views of a biocompatible enclosure 300 for a WGPU, according to an embodiment of the present invention. Enclosure 300 elevates the WGPU to allow for access to the physical ports on the underside of the chip. Enclosure 300 protects the electronic components of the WGPU from exposure to interstitial fluid while reducing immune response from the subject when implanted.

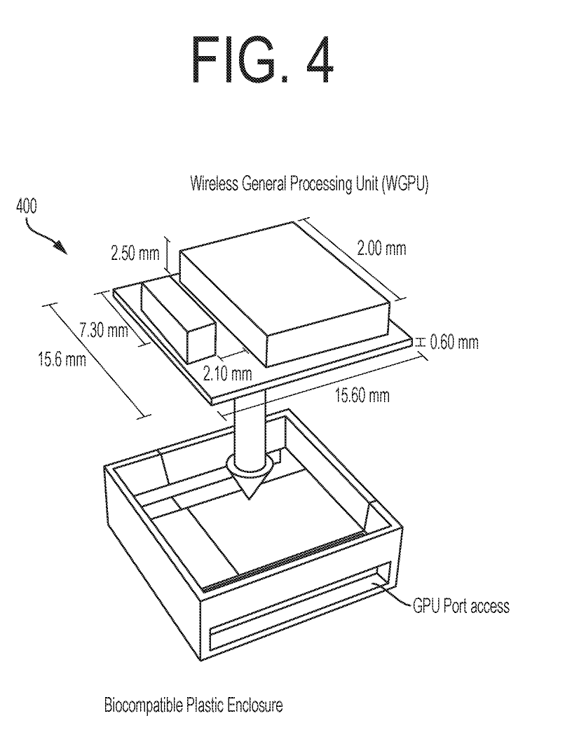

Biocompatible enclosure 300 may be made from polylactic acid or any other suitable biocompatible polymers including, but not limited to, silicone, microrenethane, poly (N-isopropyl acrylamide), polyethylene, and/or polysulfone. Enclosure 300 retains port accessibility to the general processing unit for modular connection to bioprobes and for firmware upgrades. The entire assembly may be coated with medical grade biocompatible silicone sealant or other biocompatible polymers including, but not limited to, microrenethane, polypropylene, polyethylene, polyethersulfone, and/or polysulfone to maintain separation of the electronic components from interstitial fluids upon implantation. The placement and example measurements of the WGPU inside the biocompatible enclosure are shown in separated WPGU 400 of FIG. 4.

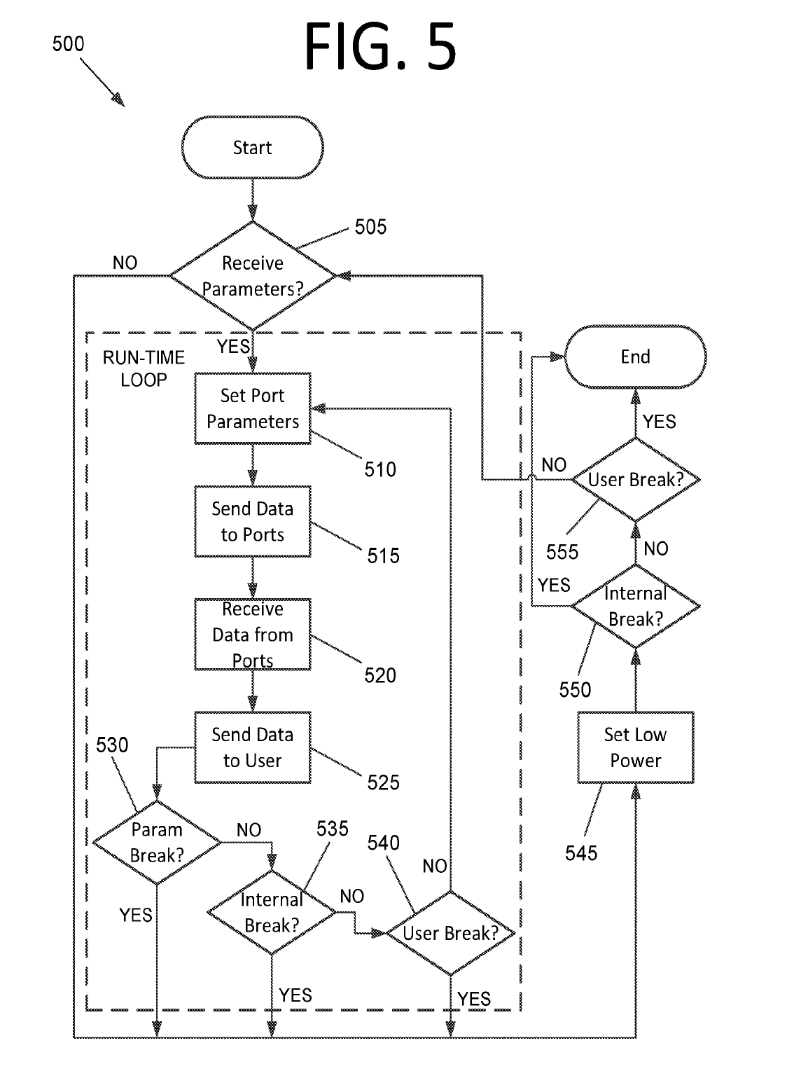

The WGPU in some embodiments may be designed with firmware to wait in low power standby until a listening event occurs, at which point the WGPU sets port parameters and initializes a loop until the user halts its operation. FIG. 5 is a flowchart 500 illustrating a process for low power standby operation of a WGPU, according to an embodiment of the present invention. The process begins with checking whether port parameters have been received by the user at 505. The user defines accessible ports depending on physically installed probes on physical ports of the WGPU. If port parameters have been received, the process proceeds into the run-time loop. If not, the process proceeds to step 545 below, where the WGPU listens for port parameter information in its low power mode until it is woken by a wireless connection including the port parameters.

If port parameters have been received at 505, the WGPU is woken and enters its run-time loop. The port parameters are set programmatically by the application at 510. The WGPU then sends digital or analog data to the ports at 515, receives digital or analog data from the ports at 520, and sends data to the user at 525. If no parameter break at 530, internal break at 535, or user break at 540 is received, the process returns to step 510 and the run-time loop continues.

However, if a parameter break at 530, internal break at 535, or user break at 540 (e.g., the user halting operation) is received, the WGPU is set to low power mode at 545. If there is an internal break at 550 or a user break at 555, the process ends. Otherwise, the process returns to step 505.

FIG. 6 is a flowchart 600 illustrating a process for setting of parameters of firmware of a WGPU via biological interface software, according to an embodiment of the present invention. The process begins with detecting WGPUs at 605 and detecting adapters at 610. If user input is provided at 615, firmware parameters are set at 620. This continues until the parameters have been input by the user. The parameters may include, but are not limited to, hardware configuration parameters, signal timing parameters, user-defined halt parameters, and/or any other suitable parameters without deviating from the scope of the invention.

At runtime, the user's WGPU port information is configured on user-defined WGPUs. If there is no parameter break at 625, internal break at 630, or user break at 635, the user's parameters are run at 640 (e.g., expected digital signals and timings are initialized). This process repeats until a break occurs, at which point, any associated external process may be run at 645, and the process ends.

A probe module in reference to the platform in some embodiments is an abstract input/output device that connects to and is controlled by the WGPU. Probe modules may receive power directly from the WGPU and/or supply their own power using their own power supply. Probe modules may be specifically designed for the specification of the end user's experiment. If the end user wishes, his or her probes may be added to the market of probes available to other possible end users in some embodiments. A library of available prebuilt probes may be provided in some embodiments to increase the usability of the platform.

In some embodiments, the WGPU ports may be assigned digital out, digital in, or analog in inside the biological interface software. Each probe should be designed to either receive 3V digital pulses and perform some action or send digital or analog signals to a listening port on the WGPU in some embodiments. Probe modules may include both stimulation and recording of optical, electrical, acoustic, and magnetic signals to and from the implanted subject in some embodiments. The probes may include, but are not limited to, an optical stimulation probe module and a 32-channel recording electrode array probe module.

An optical stimulation probe module may be a small surface mount light-emitting diode (LED) coupled with a small fiber optic core that can have a variable diameter between 100 to 300 micrometers to transmit light of a particular wavelength into or on a subject in some embodiments. FIG. 7A is an exploded perspective view illustrating an optical stimulation probe 700, according to an embodiment of the present invention. Optical stimulation probe 700 includes a high intensity surface mount LED 710 and a fiber optic core 720 coupled thereto. LED 710 and fiber optic core 720 are placed inside a biocompatible enclosure 730 and secured using optical adhesive, for instance.

In some embodiments, fiber optic core 720 and LED 710 may be directly coupled or coupled through a graded-index (GRIN) lens for higher efficiency coupling. The parts may be secured in place using transparent optical glue, depending on the wavelength of light emitted by LED 710. The entire assembly may be encased in a biocompatible polymer that may could include, but is not limited to, polylactic acid, silicone, microrenethane, poly (N-isopropyl acrylamide), polyethylene, and/or polysulfone, to ensure biocompatibility of the implant and reduce inflammatory responses.

FIG. 7B illustrates side, top, and perspective views of biocompatible enclosure 730, according to an embodiment of the present invention. Biocompatible enclosure 730 is built with a removable implantation holder (2×4 mm protruding column) in this embodiment to maintain a low profile once implanted. Probe 700 is intended to be small in some embodiments (only 5 mm×5 mm×5 mm here) once implanted and includes a control surface meant to allow the end user to precisely stereotactically place the probe on the surface of the skull, adhere the probe to the skull, then cut off the extra control surface. Probe 700 is intended to be implanted directly on the surface of the skull and secured with a biocompatible adhesive, such as one that includes polyacrylamide, but can also be used on other areas assuming the light from the LED reaches the target area.

Optical stimulation probe 700 is intended to be used in the situation when photosensitive opsins are present in an experimental subject in some embodiments. These opsins can be induced to be expressed by insertion using viral vectors or can be bred into an experimental model line by recombineering. Broadly, photosensitive opsins are used to control signaling in cells and, of experimental interest, they are coupled to ion channels that can activate or inactivate the cells whose surface to which they are attached. These actions make opsins particularly useful for scientists interested in manipulating signaling in an organism.

A 32-channel recording electrode probe module in some embodiments is an array of 32 1 MΩ up to 200-micron diameter wires connected to a 32-channel multiplexer switch and amplifier. The multiplexed signal may then be passed on to the WGPU for integration with the platform and demuxing/processing by the end user. The wire may include conductive metals and nonmetals including, but not limited to, gold, silver, tungsten, and carbon fiber. The array may be housed in a biocompatible polymer that may include, but is not limited to, polylactic acid, silicone, microrenethane, poly (N-isopropyl acrylamide), polyethylene, and/or polysulfone. The exposed portion of the wire may exit the plastic housing for direct interface with a biological recording substrate.

FIG. 8 illustrates a system 800 for recording signals from a subject 810, according to an embodiment of the present invention. A 32-channel electrode array 820 provides signals from a subject 810 (here, a mouse) to a 32-channel recording electrode probe 830. The signals contain biological information in the form of oscillating voltages from cells adjacent to electrode array 820, the wires of which are electrically exposed thereto. Implantation of electrode array 820 may include surgical removal of bone and/or epidermal tissue to gain access to the desired recording material, such as biological cells that change electrically including, but not limited to, neurons, epidermal cells, glial cells, and/or myocytes.

Probe 830 may be intended for use in the recording of neuronal spiking dynamics of ensembles of cells—a technique that is widely used among neuroscientists to decode the informational content of ensembles of neurons. Probe 830 may be used synergistically with the entire platform to stimulate regions or activate external devices based on the recording received from probe 830. Probe 830 includes a 32-channel multiplexer 832 and amplification 834 (e.g., one or more amplifiers). The signal is muxed by multiplexer 832, amplified by amplification 834, and sent to a physically connected WGPU 840 for integration with other components of the platform (e.g., biological interface software 850).

A power module may include a 3V power supply for the implanted WGPU and probes in some embodiments. The power module may be externally accessible to the experimenter and may use battery or radio frequency-induced power. If a probe requires additional power, the additional power source may be placed on the modular probe itself and be function controlled digitally by the WGPU.

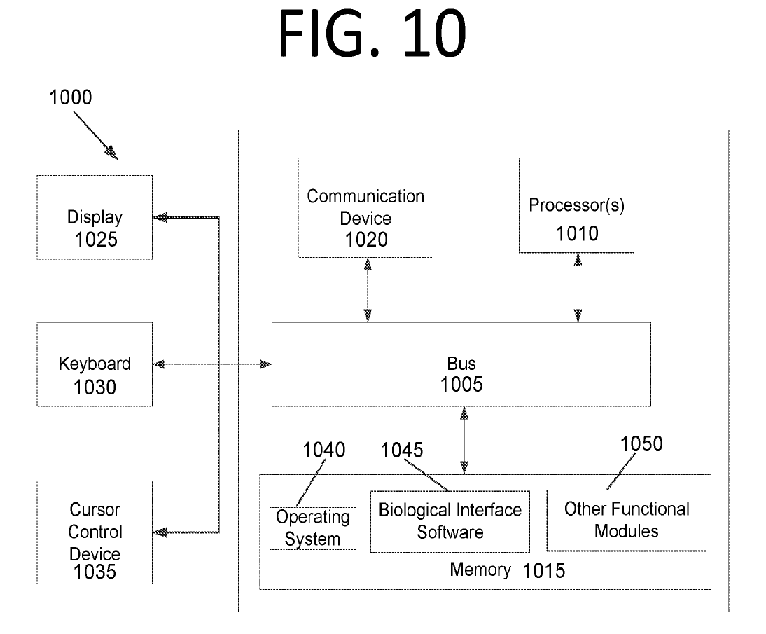

A central computer may be any computing system configured to dynamically communicate with the wireless computing core to update its existing codebase, obtain biological information from the modular probes for display to the experimenter, or send information to the computing core to dynamically activate probes. See, for example, FIG. 10.

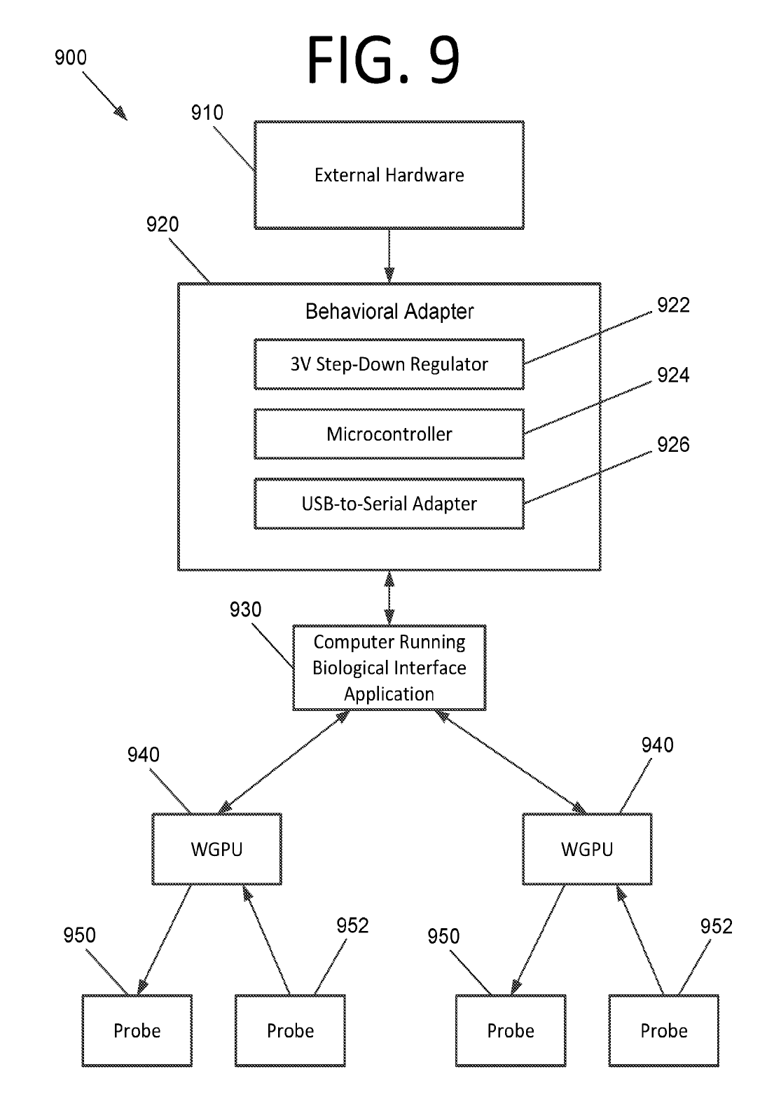

FIG. 9 illustrates a biological interface system 900 that includes a more detailed view of a behavioral apparatus adapter 920, according to an embodiment of the present invention. As with wireless biological interface platform 100 of FIG. 1, biological interface system 900 includes external hardware 910, an adapter (i.e. behavioral apparatus adapter 920), a computer 930 running a biological interface application, WGPUs 940, and probes 950, 952. However, in FIG. 9, further details regarding behavioral apparatus adapter 920 are provided.

More specifically, behavioral apparatus adapter includes a 3V 600 mA step-down regulator 922 that, in this embodiment, steps down a variable voltage signal to 3.3V for transmission to a connected microcontroller 924, and then to the biological interface application running on computer 930 via a USB connection. Controller 924 communicates with the rest of the platform using USB-to-serial adapter 926, which may be the FTDI FT232™ or any other suitable chip in some embodiments. To integrate with current behavioral standards, behavioral apparatus adapter 920 may include a Bayonet Neill-Concelman (BNC) female connector input that maps seven input pins on microcontroller 924. Behavioral apparatus adapter 920 may also expose seven 5.5V output pins that can be set high by the user using the platform software application running on computer 930. Behavioral apparatus adapter 920 may connect to an experimenter's existing behavioral apparatus (e.g., external hardware 910) to relay behavioral systems directly to computer 930 for dynamic environmentally cued control of the modular probes (e.g., probes 950, 952).

FIG. 10 is a block diagram of a computing system 1000 configured to run biological interface software, according to an embodiment of the present invention. In some embodiments, computing system 1000 may be used as computers 130 or 930 of FIGS. 1 and 9, respectively. Computing system 1000 includes a bus 1005 or other communication mechanism for communicating information, and processor(s) 1010 coupled to bus 1005 for processing information. Processor(s) 1010 may be any type of general or specific purpose processor, including a central processing unit (CPU) or application specific integrated circuit (ASIC). Processor(s) 1010 may also have multiple processing cores, and at least some of the cores may be configured to perform specific functions. Computing system 1000 further includes a memory 1015 for storing information and instructions to be executed by processor(s) 1010. Memory 1015 can be comprised of any combination of random access memory (RAM), read only memory (ROM), flash memory, cache, static storage such as a magnetic or optical disk, or any other types of non-transitory computer-readable media or combinations thereof. Additionally, computing system 1000 includes a communication device 1020, such as a transceiver, to wirelessly provide access to a communications network.

Non-transitory computer-readable media may be any available media that can be accessed by processor(s) 1010 and may include both volatile and non-volatile media, removable and non-removable media, and communication media. Communication media may include computer-readable instructions, data structures, program modules or other data in a modulated data signal such as a carrier wave or other transport mechanism and includes any information delivery media.

Processor(s) 1010 are further coupled via bus 1005 to a display 1025, such as a Liquid Crystal Display (LCD), for displaying information to a user. A keyboard 1030 and a cursor control device 1035, such as a computer mouse, are further coupled to bus 1005 to enable a user to interface with computing system 1000. However, in certain embodiments such as those for mobile computing implementations, a physical keyboard and mouse may not be present, and the user may interact with the device solely through display 1025 and/or a touchpad (not shown). Any type and combination of input devices may be used as a matter of design choice.

In one embodiment, memory 1015 stores software modules that provide functionality when executed by processor(s) 1010. The modules include an operating system 1040 for computing system 1000. The modules further include biological interface software 1045 that is configured to perform the various biological interface operations discussed herein. Computing system 1000 may include one or more additional functional modules 1050 that include additional functionality.

One skilled in the art will appreciate that a “system” could be embodied as a personal computer, a server, a console, a personal digital assistant (PDA), a cell phone, a tablet computing device, or any other suitable computing device, or combination of devices. Presenting the above-described functions as being performed by a “system” is not intended to limit the scope of the present invention in any way, but is intended to provide one example of many embodiments of the present invention. Indeed, methods, systems and apparatuses disclosed herein may be implemented in localized and distributed forms consistent with computing technology, including cloud computing systems.

It should be noted that some of the system features described in this specification have been presented as modules, in order to more particularly emphasize their implementation independence. For example, a module may be implemented as a hardware circuit comprising custom very large scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices, graphics processing units, or the like.

A module may also be at least partially implemented in software for execution by various types of processors. An identified unit of executable code may, for instance, comprise one or more physical or logical blocks of computer instructions that may, for instance, be organized as an object, procedure, or function. Nevertheless, the executables of an identified module need not be physically located together, but may comprise disparate instructions stored in different locations which, when joined logically together, comprise the module and achieve the stated purpose for the module. Further, modules may be stored on a computer-readable medium, which may be, for instance, a hard disk drive, flash device, RAM, tape, or any other such medium used to store data.

Indeed, a module of executable code could be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices, and may exist, at least partially, merely as electronic signals on a system or network.

It will be readily understood that the components of various embodiments of the present invention, as generally described and illustrated in the figures herein, may be arranged and designed in a wide variety of different configurations. Thus, the detailed description of the embodiments of the systems, apparatuses, methods, and computer programs of the present invention, as represented in the attached figures, is not intended to limit the scope of the invention as claimed, but is merely representative of selected embodiments of the invention.

The features, structures, or characteristics of the invention described throughout this specification may be combined in any suitable manner in one or more embodiments. For example, reference throughout this specification to “certain embodiments,” “some embodiments,” or similar language means that a particular feature, structure, or characteristic described in connection with the embodiment is included in at least one embodiment of the present invention. Thus, appearances of the phrases “in certain embodiments,” “in some embodiment,” “in other embodiments,” or similar language throughout this specification do not necessarily all refer to the same group of embodiments and the described features, structures, or characteristics may be combined in any suitable manner in one or more embodiments.

In addition, while the term “information” as used herein may be transmitted by a “message” and may be applied to many types of network data, such as, packet, frame, datagram, etc. The term “message” also includes packet, frame, datagram, and any equivalents thereof. Furthermore, while certain types of messages and signaling may be depicted in exemplary embodiments they are not limited to a certain type of message, and the application is not limited to a certain type of signaling.

It should be noted that reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention. In order to determine the metes and bounds of the invention, therefore, reference should be made to the appended claims.

1. A system, comprising:
a plurality of modular probes;
at least one wireless general processing unit (WGPU) operably connected to the plurality of modular probes;
at least one adapter for respective external hardware, the at least one behavioral adapter configured to relay information from the respective external hardware; and
a computing system running a biological interface application configured to process input from the at least one WGPU and the at least one adapter, the biological interface application configured to:
coordinate digital information from the external hardware received via the at least one adapter with activity of the at least one WGPU configured with one or more of the plurality of modular probes, and
receive and process the information relayed from the at least one adapter.

2. The system of claim 1, wherein the plurality of modular probes comprises at least one recording probe and at least one transmitting probe.

3. The system of claim 1, wherein the at least one WGPU is configured at runtime by the biological interface application.

4. The system of claim 1, wherein the computing system, via the biological interface application, is configured to control two or more WGPUs simultaneously, allowing multiple subjects to run simultaneously.

5. The system of claim 1, wherein the at least one WGPU comprises:
a plurality of physical ports on an underside of the at least one WGPU; and
a biocompatible enclosure that elevates the at least one WGPU to allow access to the physical ports, the biocompatible enclosure protecting electronic components of the at least one WGPU from exposure to interstitial fluid while reducing immune response from a subject when implanted.

6. The system of claim 5, wherein the at least one WGPU is coated in one or more biocompatible polymers.

7. The system of claim 1, wherein the at least one WGPU is configured to wait in a low power standby mode until a listening event occurs, at which point the at least one WGPU sets port parameters and initializes a loop until a user halts its operation.

8. The system of claim 1, wherein the biological interface application is configured to allow a user to set parameters of the at least one WGPU, the parameters comprising hardware configuration parameters, signal timing parameters, user-defined halt parameters, or any combination thereof.

9. The system of claim 1, wherein at least one of the plurality of modular probes receives power from one of the at least one WGPUs.

10. The system of claim 1, wherein at least one of the plurality of probes comprises:
a surface mount light emitting diode (LED);
a fiber core coupled to the surface mount LED; and
a biocompatible enclosure in which the surface mount LED and fiber core are placed.

11. The system of claim 10, wherein the at least one of the plurality of probes is encased in a biocompatible polymer.

12. The system of claim 10, wherein the biocompatible enclosure comprises a removable implantation holder.

13. The system of claim 1, wherein at least one of the plurality of probes is a recording probe connected to a 32-channel electrode array, the recording probe comprising:
a 32-channel multiplexer; and
one or more amplifiers, wherein
a signal received from the 32-channel electrode array is muxed by the multiplexer 832, amplified by the at least one amplifier, and sent to at least one of the at least one WGPUs for integration with other components of the system.

14. The system of claim 1, wherein the at least one adapter comprises:
a step-down regulator configured to step-down a voltage of a signal from the respective external hardware to a lower voltage;
a microcontroller configured to receive and process the stepped-down signal; and
a USB-to-serial adapter configured to receive communications from the microcontroller and relay the communications to the biological interface application of the computing system.

15. A wireless general processing unit (WGPU), comprising:
a plurality of physical ports on an underside of the WGPU; and
a biocompatible enclosure that elevates the WGPU to allow access to the physical ports, the biocompatible enclosure protecting electronic components of the WGPU from exposure to interstitial fluid while reducing immune response from a subject when implanted.

16. The WGPU of claim 15, wherein the WGPU is operably connected to a plurality of probes.

17. The WGPU of claim 15, wherein the WGPU is configured at runtime by a biological interface application.

18. The WGPU of claim 15, wherein the WGPU is configured to wait in a low power standby mode until a listening event occurs, at which point the WGPU sets port parameters and initializes a loop until a user halts its operation.

19. The WGPU of claim 15, wherein the WGPU is coated in one or more biocompatible polymers.

20. An adapter, comprising:
a step-down regulator configured to step-down a voltage of a signal from external hardware to a lower voltage;
a microcontroller configured to receive and process the stepped-down signal; and
a USB-to-serial adapter configured to receive communications from the microcontroller and relay the communications to a biological interface application of a computing system.
